(19) United States
(12) Patent Application Publication
(10) Pub. No.: US 2014/0058244 A1
(43) Pub. Date: Feb. 27, 2014

(54) PHOTOACOUSTIC MONITORING

(71) Applicant: Regents of the University of Minnesota, St. Paul, MN (US)

(72) Inventor: James C. Krocak, Minneapolis, MN (US)

(73) Assignee: Regents of the University of Minnesota, St. Paul, MN (US)

(21) Appl. No.: 13/972,571

(22) Filed: Aug. 21, 2013

Related U.S. Application Data
(60) Provisional application No. 61/691,667, filed on Aug. 21, 2012.

(51) Int. Cl. A61B 5/00; A61B 18/12

(52) U.S. Cl. CPC A61B 5/0095; A61B 18/12; A61B 5/4836
USPC 600/407; 606/34

(57) ABSTRACT

A system comprising a photoacoustic module, an ablation module, and a processor. The photoacoustic module includes a light emitter and an acoustic sensor. The acoustic sensor is configured to provide a sensor output corresponding to a modulated pressure detected in a target tissue in response to light from the emitter. The ablation module is associated with the photoacoustic module. The ablation module has an energy discharge port configured to contact the tissue at an ablation target and is configured to ablate the ablation target. The processor is coupled to the ablation module and coupled to the photoacoustic module. The processor is configured to execute an algorithm to provide an output signal indicative of the ablation progress and is configured to control the ablation module based on the sensor output.

### CLAIM OF PRIORITY

This patent application claims the benefit of priority of U.S. Provisional Patent Application Ser. No. 61/691,667, entitled “PHOTOACOUSTIC MONITORING,” filed on Aug. 21, 2012 (Attorney Docket No. 600.889PRV), which is hereby incorporated by reference herein in its entirety.

### BACKGROUND

The photoacoustic effect refers to a change in acoustic pressure arising from exposure to light energy. In particular, a tissue specimen exposed to modulated light produces modulated pressure. The modulated light can be pulsed or discontinuous. The modulated light generates temperature changes in the tissue and it is the change in temperature that produces acoustical microperturbations. The microperturbations can be oscillations detected using an acoustic sensor. The acoustic sensor can include a piezoelectric transducer or other pressure sensor.

### Overview

The present inventors have recognized, among other things, that a problem to be solved can include providing a system and method for tissue identification and for monitoring and providing rapid feedback regarding dynamic tissue modifications or regarding system modifications (such as during an ablative process).

The present subject matter provides a solution to this problem by using a photoacoustic module having a light emitter and an acoustic sensor. In one example, a processor is coupled to the photoacoustic module and is configured to determine a wavelength for the emitted light. The output from the acoustic sensor can be processed to determine a tissue type or diagnose a medical condition. In one example, the processor can provide a signal to control operation of an ablation module in real-time or near real-time. The ablation module can be configured to ablate an ablation target and the photoacoustic module can be configured to monitor a photoacoustic effect at a target tissue. In various examples, the target tissue can be coincident with, or different from, the ablation target.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 includes a system, according to one example.

FIG. 2 includes a view of a tissue.

FIGS. 3A-3D include flow charts, according to various examples.

FIGS. 4A-4C include graphs showing device performance, according to various examples.

FIG. 5 includes a graph illustrating thermal effects, according to one example.

FIG. 6 illustrates spectral content, according to one example.

FIGS. 7A and 7B illustrate devices, according to various examples.

### DETAILED DESCRIPTION

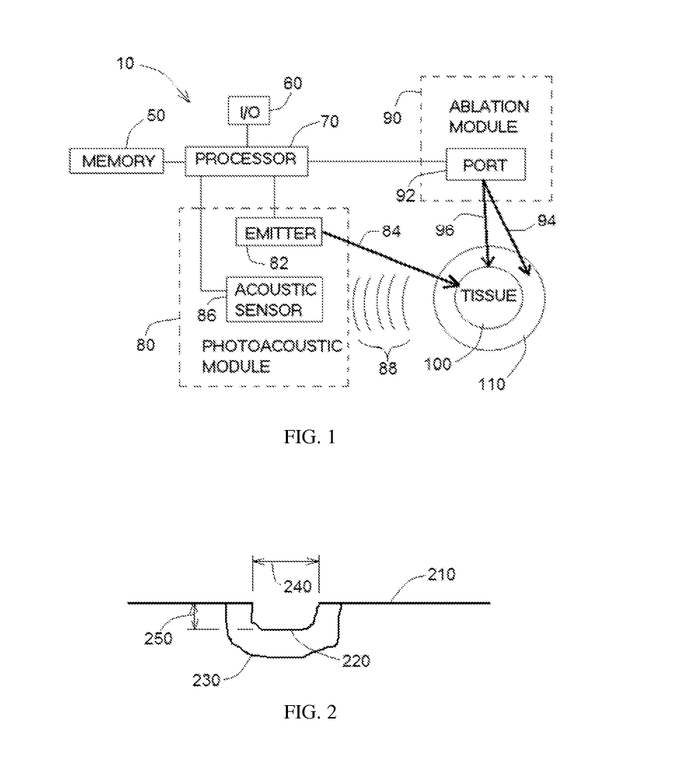

FIG. 1 illustrates system 10, according to one example. System 10 includes processor 70 coupled to photoacoustic module 80 and coupled to ablation module 90. Processor 70 is also coupled to memory 50 and input/output (I/O) 60. In the example shown, the interconnections are represented by lines that can denote either a wired connection or a wireless communication channel.

Photoacoustic module 80 includes emitter 82 and acoustic sensor 86. Emitter 82 can include an optical (light) emitter, examples of which can include a light emitting diode (LED), a laser, a fiber optic channel, or other light channeling or generating material. Acoustic sensor 86 can include a piezoelectric element, a microphone, a pressure sensor, or other sensor configured to generate an output signal corresponding to pressure variations or acoustical energy.

Ablation module 90 includes port 92. Port 92 can include a transducer configured to emit ultrasonic energy, light energy (such as a laser), thermal energy, radio frequency (RF) energy, or chemical energy. Port 92 can be configured to emit a focused beam of energy suitable for localized ablation, heating (such as cautery or coagulation) or other application of energy. In one example, port 92 is configured to directly couple with target tissue 100. Direct coupling entails an uninterrupted pathway between the output of port 92 and tissue 100. Indirect coupling might include a transfer medium or a balloon.

In various examples, ablation module 90 is configured to includes an RF electrode, an ultrasound transducer, an ultrasound array (such as a HIFU array), an irreversible electroporation probe, a sonoporation probe, a laser, a cryoablation probe, a vapor ablation probe, a chemical ablation probe, and a DC current ablation member or other device configured to cause tissue modification or destruction.

As shown in FIG. 1, port 92 is configured to emit energy as indicated by arrow 96 directed to target tissue 100. In addition, emitter 82 is configured to emit optical energy at target tissue 100 as indicated by arrow 84. In response to the effects of the emitted energy from emitter 82, target tissue 100 is the source of localized heating and pressure waves 88. Pressure waves 88 are detected by acoustic sensor 86.

Processor 70 is configured to execute an algorithm to perform a method as described elsewhere in this document. Memory 50 can provide storage for the algorithm, storage for data (such as a database), operating parameters, measured data, calculated data, and other information. I/O 60 can include a keyboard, a monitor (display), a speaker, a microphone, a cursor control (such as a mouse), a network interface, a printer, or other device suitable for providing data to processor 70 or suitable for rendering data from processor 70.

Processor 70 can be integrated into a unitary device (here denoted as system 10) along with the photoacoustic module and the ablation module or may be external to the device. Processor 70 can be configured to interpret the generated photoacoustic signals and translating them into normalized, standardized, or other readily interpretable parameters or values.

In the example illustrated, emitter 82 is configured to direct optical energy to target tissue 100 and, as shown at arrow 96, port 92 is configured to emit focused ablative energy, also on target tissue 100. In this example, the target tissue and the ablative tissue are coincident.

In addition, in the example shown, port 92 can be configured to emit focused energy on nearby tissue 110. Nearby tissue 110 is the ablative target and in this example, the radiated pressure waves 88 are responsive to thermal energy from both the target tissue (target with respect to emitter 82) an ablative target (with respect to ablative energy from port 92, along arrow 94).

Port 92 can be configured to direct ablative energy along a selected path. Path selection can include modulating or controlling an electrical signal delivered to port 92. In one example, a path is selected by physically manipulating alignment of a transducer relative to the site of the target tissue.

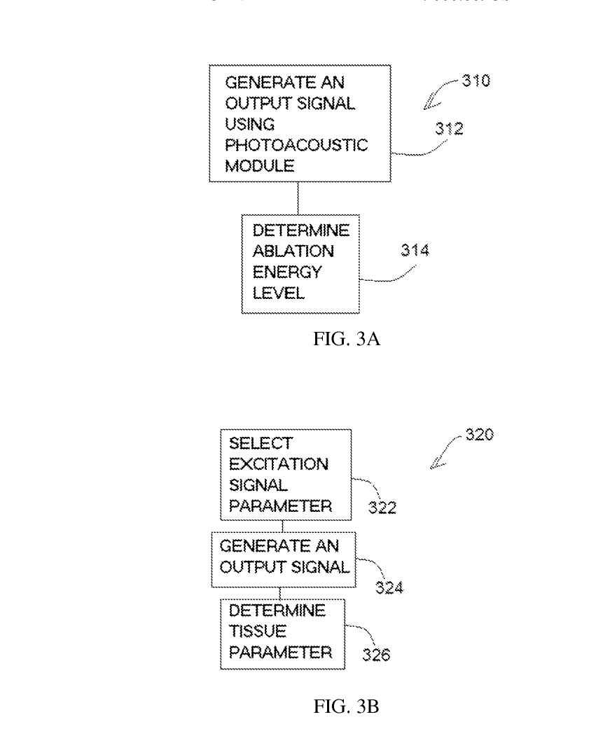

FIG. 2 illustrates a cross-sectional view of tissue 210. Tissue 210 includes a lesion formed by, for example, an ablation operation. In the figure, ablation region 220 has a width denoted by dimension 240 and a depth denoted by dimension 250. Region 230, in the example shown, is adjacent ablation region 220. Region 230 can include a heat-affected zone. Emitter 82 (FIG. 1) can be configured to optically excite region 230 and an output signal from acoustic sensor 86 (FIG. 1) can indicate progress of lesion formation in ablation region 220. Dimension 240 and dimension 250 can represent the size of a lesion formed by ablation.

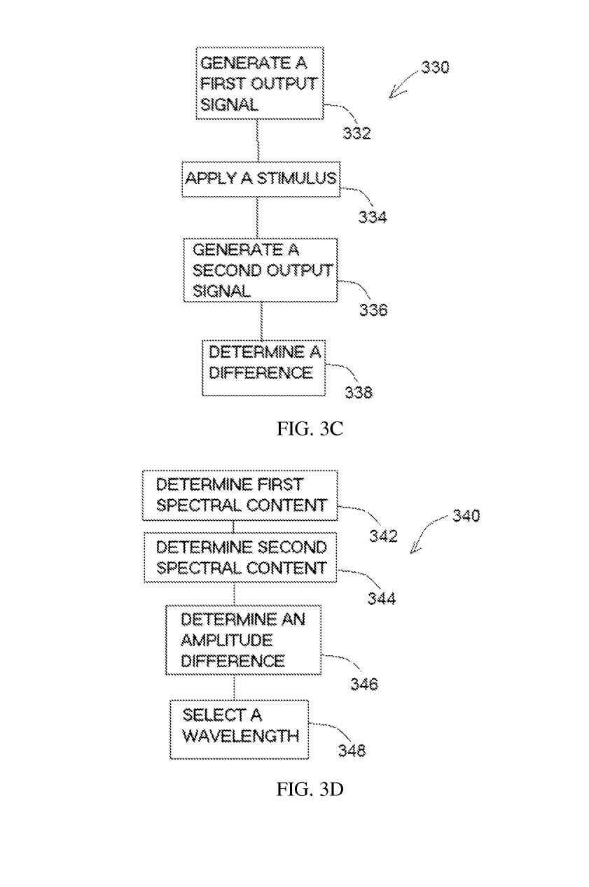

FIG. 3A illustrates method 310 corresponding to one example of the present subject matter. Method 310 includes, at 312, generating an output signal using a photoacoustic module. The output signal from the photoacoustic module can include an electrical signal corresponding to a modulated pressure wave.

Method 310 includes, at 314, determining an ablation energy level. The ablation energy level can be configured to achieve a selected size of lesion or elevate a target tissue temperature to a predetermined level.

An algorithm executing on a processor (such as processor 70) can determine the ablation energy level. In various examples, the energy level can be generated in real-time or near real-time. Near real-time is a time duration that is sufficiently short that modulations or adjustments in the ablation energy level are less than approximately two seconds after generating the output signal.

FIG. 3B illustrates method 320 corresponding to one example of the present subject matter. Method 320 includes, at 322, selecting an excitation signal parameter. The excitation signal parameter can include a frequency, a wavelength, or amplitude for emission of optical energy by emitter 82 of a photoacoustic module and directed to a site of a target tissue.

Method 320 includes, at 324, generating an output signal corresponding to a detected pressure wave from the target tissue.

Method 320 includes, at 334, determining a tissue parameter. The tissue parameter is associated with the target tissue and can indicate, for example, that the target tissue is cancerous or that the target tissue includes nerve tissue. An algorithm executing on a processor can be configured to determine the tissue parameter using, for example, a look up table, a database, a neural network, artificial intelligence, or based on a comparison with archived data for a patient associated with the particular target tissue or for a patient population.

FIG. 3C illustrates method 330 corresponding to one example of the present subject matter. Method 330 includes, at 332, generating a first output signal using a photoacoustic module. The first output signal from the photoacoustic module can include an electrical signal corresponding to a modulated pressure wave under conditions including a particular excitation from an emitter.

Method 330 includes, at 334, applying a stimulus. The stimulus can include conducting an intervention, performing a surgical procedure, performing ablation, adjusting a pressure or a temperature or other stimulus.

Method 330 includes, at 336, generating a second output signal using the photoacoustic module. The second output signal from the photoacoustic module can include an electrical signal corresponding to a modulated pressure wave under conditions corresponding to those used for generating the first output signal.

Method 330 includes, at 338, determining a difference as to the first output signal and the second output signal. In one example, this includes calculating amplitude differences on a per wavelength (or per frequency) basis.

In addition, determining the difference can include executing an algorithm to perform the calculation. Furthermore, the algorithm can be configured to generate an output based on a calculated maxima or minima.

FIG. 3D illustrates method 340 corresponding to one example of the present subject matter. Method 340 includes, at 342, determining a first spectral content using a photoacoustic module. The spectral content can be determined by sweeping an emitted frequency or sweeping an emitted wavelength and generating an output signal using an acoustic sensor of a photoacoustic module. The output signal from the photoacoustic module can include an electrical signal corresponding to a modulated pressure wave. In one example, the first spectral content is determined based on a first target tissue site.

Method 340 includes, at 344, determining a second spectral content using a photoacoustic module. The spectral content can be determined by sweeping an emitted frequency or sweeping an emitted wavelength and generating an output signal using an acoustic sensor of a photoacoustic module. The output signal from the photoacoustic module can include an electrical signal corresponding to a modulated pressure wave. In one example, the second spectral content is determined based on a second target tissue site, in which the first target tissue site and the second target tissue site are different. For example, the first target tissue site can be at a specific location corresponding to cancerous cell and the second target tissue site can be at a nearby location corresponding to noncancerous cells.

Method 340, at 346, includes determining an amplitude difference based on the first spectral content and the second spectral content. The difference can be determined by an algorithm executing on a processor coupled to the photoacoustic module.

Method 340, at 348, includes selecting a wavelength based on the calculated amplitude difference. In one example, this includes selecting a maximum differential.

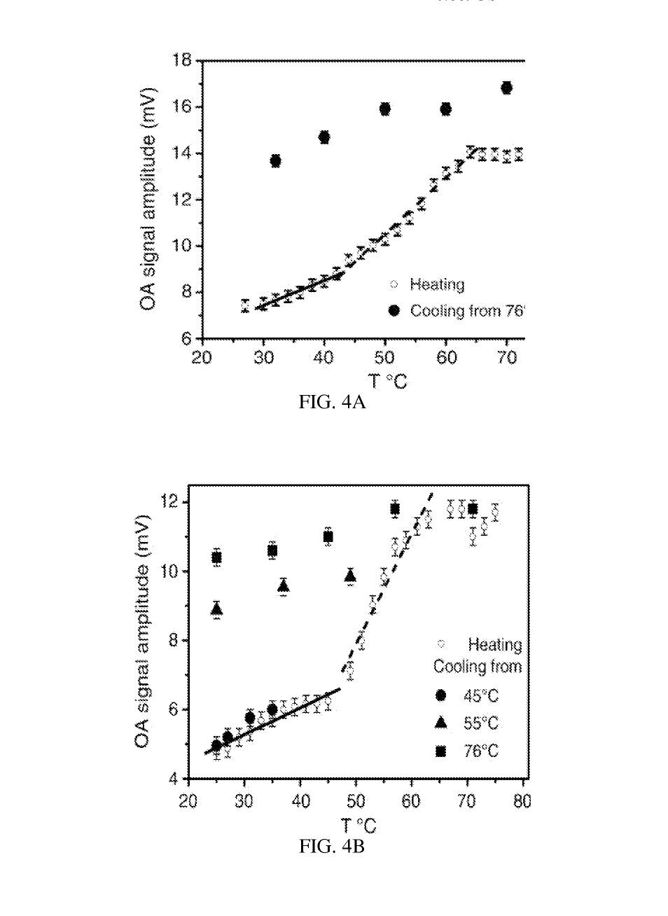

FIGS. 4A, 4B, and 4C, illustrate data corresponding to a liver, to a muscle, and to fat, according to various examples. The illustrated data demonstrates tissue differentiation based on an output signal from a photoacoustic module. In the examples shown, the photoacoustic signals, generated for three different tissues (liver, muscle, and fat), are illustrated as a function of temperature. In addition to illustrating tissue differentiation based on the photoacoustic effect, this data illustrates the ability of the photoacoustic effect to provide data relating to tissue temperature. In the figures, the output signal amplitude (in millivolts) is shown on the ordinate and the swept temperature is shown on the abscissa.

An algorithm executed on a processor can be configured to discern the tissue type (or a medical condition associated with the tissue) based on one or more parameters elicited from the illustrated data. For example, the data shown varies according to slope, amplitude, and curve morphology.

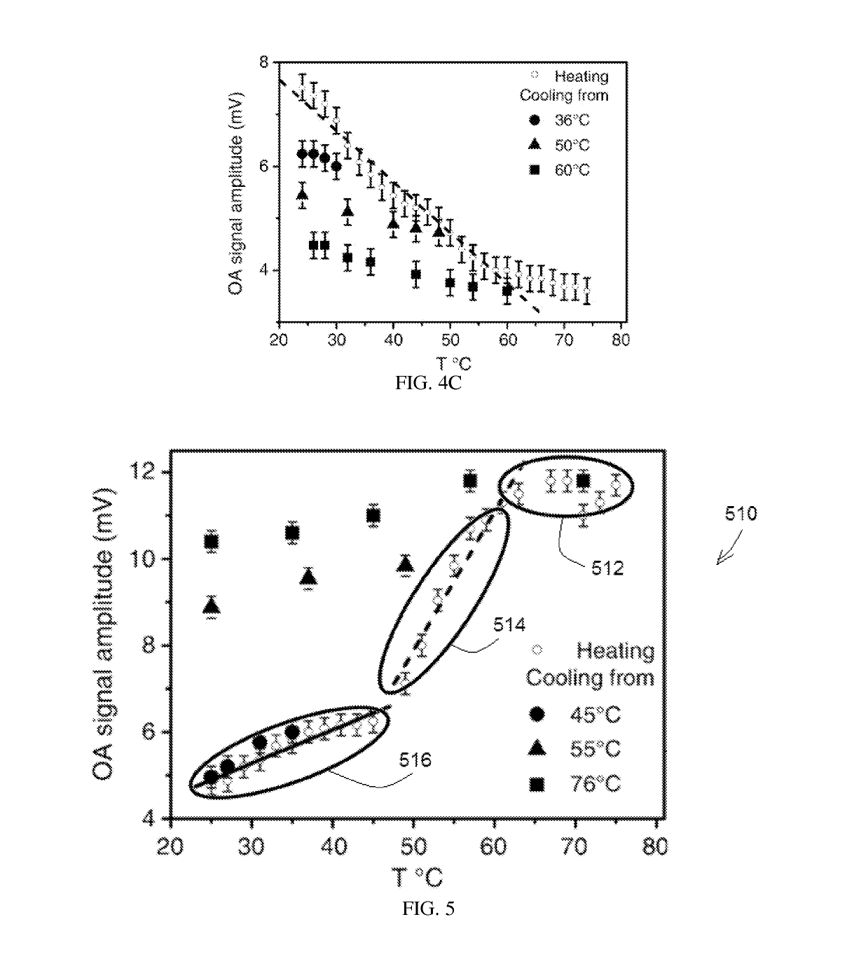

FIG. 5 illustrates analysis of a photoacoustic signal versus temperature for muscle tissue. In the figure, region 516 illustrates a low slope, pre-coagulation regime. In region 516, the output signal is generally linear. Region 514 illustrates a comparatively high slope, coagulation regime. The phrase “low slope” and “high slope” are indicative of the absolute value of the slope, and the obtained values may be either positive, negative, or zero. In region 514, tissue coagulation is actively occurring and the output signal is generally linear. In region 512, the output signal has reached a plateau and tissue coagulation has reached completion. Region 512 is a readily identifiable endpoint for tissue ablation. In other words, when the plateau behavior is recognized (for example, by a processor executing an algorithm) tissue ablation procedures can be terminated.

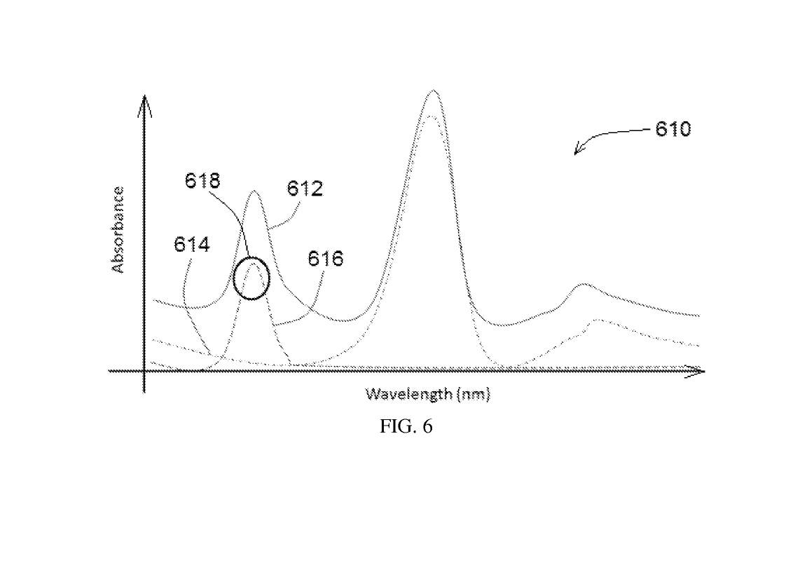

FIG. 6 illustrates an example of wavelength selection. In this example, spectral content is depicted on common coordinate system 610. Spectra of the target tissue is illustrated at 612. Nearby healthy tissue is shown at 614. The difference spectra (subtracted) are illustrated at 616. A maximum of the subtracted spectra will provide a wavelength that will yield a maximized photoacoustic signal of the target tissue relative to nearby tissue. In the example shown, the maximum or peak signal difference occurs at the wavelength (or frequency) denoted by circle 618.

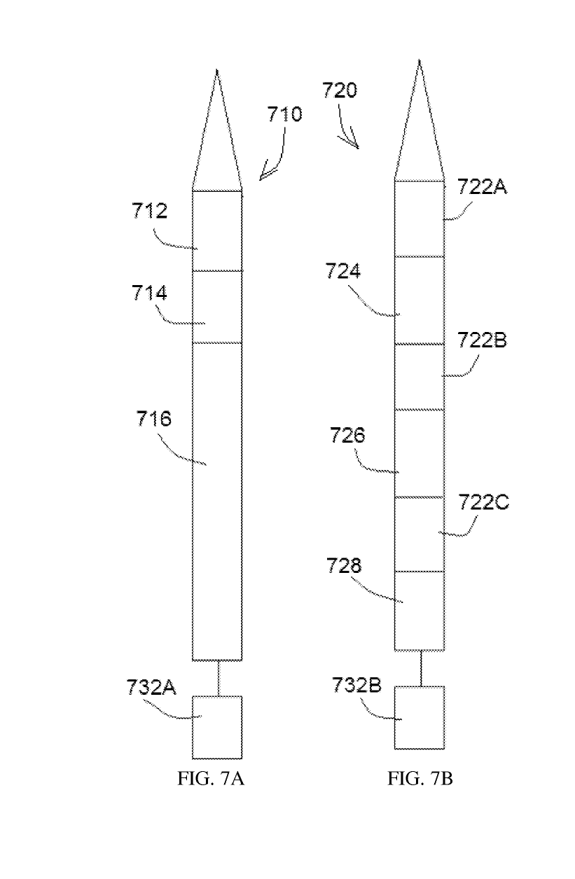

FIGS. 7A and 7B illustrate models having a cylindrical form and corresponding to different examples of the present subject matter. FIG. 7A illustrates a model of device 710 having acoustic sensor 712 and emitter 714 each of which are coupled to processor 732A. In device 710, a single acoustic sensor 712 is used in conjunction with emitter 714, however other examples are also contemplated including, for example, a plurality of light emitters 714 or a plurality of acoustic sensors 712. In addition, the relative placement of light emitter 714 and acoustic sensor 712 can be configured so that either are distal, proximal, or overlapping, so long as they are positioned such that they can work cooperatively. In this manner, the pressure waves generated by the light pulses (from emitter 714) can be detected by acoustic sensor 712. Body 716 provides mechanical stability and a housing for other electronics (power supply, telemetry, guidance or other circuitry).

FIG. 7B illustrates a model of device 720. In the example shown, device 720 includes acoustic sensor 724. Acoustic sensor 724 can include a flexible piezoelectric film, and can be focused or unfocused, and can be wrapped circumferentially around device 720 or may be directed away from only a portion of device 720. In other examples, a plurality of acoustic sensors 724 are distributed along a length of device 720.

Device 720 includes light emitter 726 which can include a laser, a fiber optic channel, or other light channeling or generating material and elements that are capable of delivering the target wavelength of light to the desired tissue at a sufficient power. In other examples, a plurality of emitters 726 are distributed along a length of device 720.

Device 720 includes ablative elements 722A, 722B, and 722C. Ablative elements 722A, 722B, and 722C are configured to perform either thermal or non-thermal ablation. Device 720 includes processor 732B. Processor 732B can be integrated into device 720 or can be external to device 720 and in communication with device 720. Processor 732B can be configured to interpret the generated photoacoustic signal and translating them into normalized, standardized, or other readily interpretable parameters or values.

Body 728 provides mechanical stability and a housing for other electronics (power supply, telemetry, guidance or other circuitry).

An ablative procedure can be used to treat atrial fibrillation and hypertension. Ablation entails forming lesions or by modifying tissue properties, damaging or killing tissue, or disrupting neurological pathways.

An example of the present subject matter can be used to clinically monitor and assess lesion formation in terms of size, depth, and tissue temperature in real-time, or in near real-time. Historically, medical professionals have relied on time correlations, and consequently, may form lesions that are too large or too small, either of which can have detrimental side effects or reduce the overall efficacy or patency of the procedure, and in turn increase the financial strain on the medical system.

One example provides tissue identification using the photoacoustic effect. Tissue identification can facilitate distinguishing cancer, nerves, or other tissues, as well as for monitoring ablation of tissue in real-time, near real-time, continuously, or discontinuously. An example can be used to identify a cancerous region within a patient, and following a medical procedure, such as ablation, an example of the present subject matter can assess eradication or presence of viable cancer cells.

In addition, lesion formation can be monitored in areas surrounding nerve fibers during ablative procedures, such as renal denervation, to ensure patency following the procedure, while enabling minimization of the overall lesion size. Lesion penetration depth can be assessed, which can be important for ablative procedures of thin tissues, such as for treatment of Barrett's esophagus.

The photoacoustic effect utilizes pulsed, or discontinuous, transmission of light waves to deliver energy to a tissue and in turn cause microperturbations in temperature within the tissue. These small temperature fluctuations cause oscillations within the tissue, resulting in formation of pressure gradients within the tissue. The pressure gradients can then be measured, monitored, and detected, using various sensors. For example, a piezoelectric transducer or other acoustic sensor element can be used.

The photoacoustic effect can be used for tissue differentiation and selection. Different tissue and different cell types preferentially absorb select, specific wavelengths of light, and consequently, different cell and tissue types absorb light of different wavelengths. As such, if a particular tissue type is being targeted, intrinsically unique or preferential absorption wavelengths can be selected to deliver elevated levels of energy to specific tissues or cells that maximally absorb that wavelength.

In some examples, such as for cancer identification, cells from a biopsy or other tissue (or cell) extraction mechanism, can be analyzed (ex-vivo or in-vivo) using spectral analysis (spectrophotometers) to identify preferential absorption wavelengths of the cancer cells relative to nearby or healthy cells and tissue.

The emitted light can be specifically tuned to a preferential wavelength to provide better visualization or identification of cancer cells. This approach can also be used with nerve fibers or other tissue classes.

For certain tissue classes, the physiological and spectral properties of the target tissue are relatively consistent across a population of patients, and accordingly, tissue property correlations can be utilized in addition to, or in place of, patient-specific spectral analyses.

In one example, a tissue-monitoring device includes one or more light emitters and one or more acoustic sensors in cooperative arrangement for identifying tissue or for monitoring dynamic changes within tissue.

Cooperative arrangement provides that the light emitters and the acoustic sensors are in close proximity to each other, either distally, proximally, or overlapping with each other. In operation, the at least one light emitter is configured to emit light of a particular wavelength to confer some tissue or cellular specificity and the acoustic sensor is configured to obtain information relating to the pressure fluctuations generated in the tissue due to absorption of light.

Excitation of the tissue by a light emitter either can target a portion of the tissue, or can be broadly applied to the whole tissue. The light can initially be applied in a single direction and then circumferentially translated or swept in a geometric pattern through other regions of the tissue.

The excitation (light energy) can be either provided via a beam or broadly applied in a non-focused manner. The processor is configured to conduct analysis and, in one example, is configured to interpret the signals detected by the acoustic sensor. Output from the processor can include information regarding the current state of the analyzed tissue, such as temperature, water content, protein content, or other physiological properties, rheological parameters, viscosity, or other parameters that can be extracted via photoacoustic analyses. Translation of this output can be encoded in a single parameter or a multiple parameter output. In one example, the processor is configured to generate a 3-dimensional representation of the analyzed region.

An example of the present subject matter includes a tissue ablation device having one or more light emitters, one or more ablative members, and one or more acoustic sensors in cooperative arrangement.

Light is emitted into the surrounding tissue and the generated temperature or pressure fluctuations can be observed or monitored by the acoustic sensors.

Modification of the tissue via the ablative device can occur prior to, concomitantly with, or following, observation of the surrounding tissue with the light emitter and acoustic sensor via the photoacoustic effect. Changes within the tissue properties, which occur during lesion formation (such as heating, freezing, or poration) can be observed continuously, discontinuously, in real-time, or in near real-time.

In one example, the processor provides an output indicative of a measure of the ablative process.

If thermally destructive means are utilized, such as with RF or HIFU, this measure can include temperature. In one example, the processor generates an output to indicate completion of the ablative process. Completion can be indicated by coagulation within the targeted tissue and can be visualized by temporally stabilized photoacoustic output, as detected by an acoustic sensor element.

If a non-thermal destruction/modification device is utilized, such as irreversible sonoporation or irreversible electroporation, the output can relate to the fraction of viable cells that remain in the target region or tissue. In some poration processes, external media enters cells causing intracellular changes, and in turn, cell death. External agents may be infused into the tissue prior to poration or ablation, such that following poration, these exogenous agents enter the cell, thereby further modifying the intracellular environment, such that intracellular changes are more readily detectable via the photoacoustic effect. This process can be achieved via the exogenous agents or specifically reacting with intracellular components, such that the absorption spectrum of the successfully porated cells is shifted relative to non-effected cells. In one example, the spectral shift is such that the porated cells absorb light of a particular wavelength that unaffected cells do not absorb or only minimally absorb. Such agents can include select reactive species that can bind or react with intracellular components, antibodies, or other compounds with specific intracellular activity, including, but not limited to destruction via pH modification, such as with base or acid, fluoroscopic modification, such as through fluorescent probes or compounds, or spectrally distinct compounds, that actively react with intracellular compounds due to the elevated levels of pharmacologically active agents within the cells, including but not limited to proteases and enzymes that have biological activity. One example includes a green fluorescent protein chimera that is inactive (non-fluorescent) in its native configuration and fluoresces when cleaved by intracellular proteases, such as tryp sin.

Exogenous agents can be actively driven into the targeted tissue via a variety of processes, including for example, sonophoresis, electrophoresis, pressure gradients, temperature gradients, or other physiological gradients that can induce a velocity vector, mass transport, or motion of exogenous species within a targeted tissue.

One example includes a method for observing dynamic tissue changes. Tissue and cells change in response to a variety of external and internal factors, including temperature, pressure, and electrical energy density. These changes can include modification of water or protein content, over or under expression of specific proteins, denaturation of proteins, changes in viscoelastic properties, and compliance.

These modifications correspond to altered spectral properties. The target tissue or cell, after modification, will have a different absorption spectrum relative to the unaffected, or unaltered, specimen. The change in spectral properties corresponds to different wavelengths of light to deliver increased energy load to these specific tissues.

Accordingly, manipulation of the wavelength of light used for the photoacoustic effect can confer tissue selectivity or specificity. Changes in spectral properties during external manipulation of the environmental conditions can be used to track dynamic intra-tissue properties in response to processes (including thermal and non-thermal ablation), such as ablative procedures performed with RF energy or via poration processes, such as irreversible electroporation.

In one example, a target tissue is first irradiated with light energy of a specific wavelength that is preferentially absorbed relative to nearby or adjacent tissues. Irradiation can be either continuous or with discontinuous pulses of the target wavelength. Following irradiation, an acoustic sensor can be configured to sense micro pressure fluctuations originating from within the targeted tissue. These pressure fluctuations can be analyzed in context of the duration and periodicity of the pulsed light, and, in concert with the acoustic resistance and velocity properties of the target tissue, signal depth can be determined, based on the amount of time between when the light pulses were delivered and when the corresponding acoustic signal was received. Other signal characteristics, such as signal amplitude, peak frequency shift, and frequency roll-off signature, can be illustrative of the state of the targeted physiological property, such as temperature, coagulation state, etc.

After analysis, the tissue can be subjected to an external influence, such as temperature fluctuations. The photoacoustic monitoring process can be repeated intermittently to obtain a discontinuous depiction of how the physiological properties are changing, or the process can be continuous, to obtain a smooth function of the physiological change. An illustrative example is for thermal ablation/coagulation of tissue, where the amplitude of the resulting acoustic signal increases with increasing temperature, which is illustrative of overall coagulation state. When the coagulation process is completed, the resulting amplitude of the generated photoacoustic signal stabilizes, regardless of overall tissue temperature, which is suggestive of a fully coagulated tissue.

In one example, a method is configured for improved tissue selectivity and specificity. A method includes identifying a target tissue or cell type (such as cancer, such as HER2+ breast carcinoma). The method includes subjecting the specific cell (or tissue) to a spectral analysis, such as with a spectrophotometer, generally with a wavelength range from 10-10,000 nm, or wavelengths that can be generated by spectraphotometric light sources or lasers. This can be achieved either in vivo or ex vivo.

An example (ex vivo) includes collecting a tissue sample by biopsy or other tissue extraction method. If specific cells are targeted, then these cells can be identified via FACS, ELISA, or other biological tools. In addition, cells or tissue that are in near proximity to the target tissue or cells (this may include one or more tissue or cell classes) are obtained, and a similar spectral analysis can be performed. In both situations, a spectrum (absorbance intensity as a function of wavelength), is generated. To improve tissue selectivity, a wavelength of light (for use by the photoacoustic module emitter) is selected such that the target cell (or tissue) absorbs at that wavelength and the nearby tissue (or cells) do not absorb (or minimally absorb) the wavelength.

In one example, to improve the generated photoacoustic signal of the target tissue relative to the nearby healthy tissue, the two (or more) spectra are subtracted. In this case, the spectrum of the nearby tissue (or cells) is subtracted from the spectrum of the target tissue (or cells). The optimized wavelength will be the maximum of the subtracted spectra.

Consider an example configured for monitoring dynamic tissue properties during thermal ablation.

In his example, a Nd:YAG laser, with wavelength (□) of 1064 nm, intensity of 2 mJ at a target region within the targeted tissue, with a pulse width of 12 ns, pulse repetition of 50 Hz, and 128 cycles can be used to obtain real time temperature data relating to muscle tissue during a thermal ablation process. Temperature data can be obtained in real time, with temporal resolution of approximately 0.02 seconds. Temperature values are based on calibration curves and photoacoustic output, and are accurate to approximately 1 degree Celsius. Data from this experimental setup can be represented by FIGS. 4A-4C and FIG. 5. In these figures, the ability of the photoacoustic effect to differentiate tissue class is illustrated in FIGS. 4A-4C and the ability of the photoacoustic effect to generate information that is correlated with temperature is illustrated in FIGS. 4A-4C and FIG. 5. The ability to detect an ablation endpoint is illustrated in FIG. 5.

As used herein, the wavelength and frequency are related by a velocity. Assuming the velocity is substantially constant, the reference to wavelength can be interchanged with a reference to frequency.

### Various Notes & Examples

Example 1 can include a system having a photoacoustic module, an ablation model and a processor. The photoacoustic module can include a light emitter and an acoustic sensor. The acoustic sensor can be configured to provide a sensor output corresponding to a modulated pressure detected in a target tissue in response to light from the emitter. The ablation module is associated with the photoacoustic module. The ablation module has an energy discharge port configured to contact the tissue at an ablation target. The ablation module is configured to ablate the ablation target. The processor is coupled to the ablation module and is coupled to the photoacoustic module. The processor is configured to execute an algorithm to provide an output signal indicative of ablation progress. The processor is configured to control the ablation module based on the sensor output.

Example 2 can include, or can optionally be combined with the system of Example 1 to optionally include wherein the sensor output corresponds to size, depth, or temperature of a lesion at the ablation target produced by the ablation module.

Example 3 can include, or can optionally be combined with system of Example 1 to optionally include wherein the ablation target and the target tissue are coincident.

Example 4 can include, or can optionally be combined with the system of Example 1 to optionally include wherein the photoacoustic module provides a near real-time or real-time feedback signal corresponding to a lesion at the ablation target.

Example 5 can include, or can optionally be combined with the system of Example 1 to optionally include wherein the ablation module includes at least one of a laser, a radio frequency (RF) probe, an ultrasonic emitter, an irreversible electroporation probe, a sonoporation probe, an ablative laser, a cryoablation probe, a vapor ablation probe, a chemical ablation probe, or a DC current ablation member.

Example 6 can include, or can optionally be combined with the system of Example 1 to optionally include wherein the discharge port is configured to emit the energy directly to the target tissue.

Example 7 can include or use subject matter such as generating an output signal using a photoacoustic module and determining an ablation energy level. The output signal corresponds to a sensor output associated with a modulated pressure detected in a target tissue in response to light from an emitter of the photoacoustic module. The output signal corresponds to a physical parameter of a lesion in the target tissue. The subject matter includes determining an ablation energy level for an ablation module. The ablation energy level is based on the output signal. The ablation energy level is determined by a processor executing an algorithm. The ablation energy level corresponds to ablation energy for delivery directly to the tissue.

Example 8 can include or can optionally be combined with the method of Example 7 to optionally include wherein the physical parameter of the lesion corresponds to at least one of size, depth, and temperature.

Example 9 can include or can optionally be combined with the method of Example 7 to optionally include wherein determining the ablation energy level includes modulating the ablation energy level.

Example 10 can include or can optionally be combined with the method of Example 7 to optionally include identifying a diseased condition of the target tissue based on the output signal.

Example 11 can include or can optionally be combined with the method of Example 7 to optionally include identifying a tissue type of the target tissue based on the output signal.

Example 12 can include or can optionally be combined with the method of Example 7 to optionally include determining a signal parameter corresponding to the light from the emitter, the signal parameter determined based on a tissue type for the target tissue.

Example 13 can include or can optionally be combined with the method of Example 7 to optionally include wherein the signal parameter includes at least one of a wavelength and a frequency.

Example 14 can include or can optionally be combined with the method of Example 7 to optionally include wherein determining the signal parameter includes conducting a biopsy, conducting a tissue extraction method, conducting a cell extraction method, conducting spectral analysis, or accessing a database.

Example 15 can include or can optionally be combined with the method of Example 7 to optionally include wherein determining an ablation energy level includes at least one of selecting a temperature for ablation, selecting a coagulation detection threshold, selecting a timing parameter of the ablation, modulating the ablation energy level to achieve a predetermined temporal stability in the output signal, calculating a measure of viable cells, or determining a shift in a spectral content.

Example 16 can include or can optionally be combined with the method of Example 7 to optionally include delivering an exogenous agent to the target tissue.

Example 17 can include or can optionally be combined with the method of Example 7 to optionally include wherein delivering the exogenous agent includes delivering a fluorescent protein or other compound having a distinctive spectral absorbance feature.

Example 18 can include or use subject matter including selecting an excitation signal parameter. The method can include generating an output signal and determining a tissue parameter. Generating an output signal includes using a photoacoustic module. The output signal corresponds to a sensor output associated with a modulated pressure detected in a target tissue in response to light from an emitter of the photoacoustic module. The light is modulated according to the excitation signal parameter. The method can include determining a tissue parameter corresponding to the target tissue and based on the output signal.

Example 19 can include or can optionally be combined with the method of Example 18 to optionally include wherein the tissue parameter is associated with a tissue type.

Example 20 can include or can optionally be combined with the method of Example 18 to optionally include wherein the tissue parameter corresponds to a dynamic tissue characteristic.

Example 21 can include or can optionally be combined with the method of Example 18 to optionally include wherein the light from the emitter is delivered in a beam.

Example 22 can include or can optionally be combined with the method of Example 18 to optionally include wherein the light from the emitter is unfocused.

Example 23 can include or can optionally be combined with the method of Example 18 to optionally include wherein the light from the emitter is swept in a range about the signal parameter.

Example 24 can include or can optionally be combined with the method of Example 18 to optionally include wherein the light from the emitter is held constant at the signal parameter.

Example 25 can include or can optionally be combined with the method of Example 18 to optionally include wherein determining the tissue parameter includes determining a pressure.

Example 26 can include or can optionally be combined with the method of Example 18 to optionally include wherein determining the tissue parameter includes determining spectral content.

Example 27 can include or use subject matter such as a method such as can include generating a first output signal using a photoacoustic module. The first output signal corresponds to a sensor output associated with a modulated pressure detected in a target tissue in response to light from an emitter of the photoacoustic module, the light modulated according to a first excitation signal parameter. The method includes applying a stimulus to the target tissue. The method includes generating a second output signal using the photoacoustic module. The second output signal corresponds to the sensor output associated with a modulated pressure detected in the target tissue in response to light from the emitter. The light is modulated according to a second excitation signal parameter. The method includes determining a difference output based on a comparison of the first output and the second output.

Example 28 can include or can optionally be combined with the method of Example 27 to optionally include wherein determining the difference includes determining a change in amplitude.

Example 29 can include or can optionally be combined with the method of Example 27 to optionally include wherein determining the difference includes determining a change in frequency.

Example 30 can include or can optionally be combined with the method of Example 27 to optionally including determining a time difference between the first output signal and the second output signal.

Example 31 can include or use subject matter such as a method such as can include or use determining spectral content corresponding to tissue at a target site, determining spectral content corresponding to tissue at an offset site, determining an amplitude difference, and selecting a wavelength based on an amplitude difference. The method includes determining spectral content corresponding to tissue at a target site. The spectral content includes amplitude as a function of wavelength. The wavelength is swept over a predetermined range. The amplitude is determined using a photoacoustic module. The photoacoustic module includes a light emitter and an acoustic sensor. The acoustic sensor is configured to provide a sensor output corresponding to a modulated pressure detected in a target tissue in response to light from the emitter. The light from the emitter corresponds to the selected wavelength. The method includes determining spectral content corresponding to tissue at an offset site. The offset site is displaced from the target site by a distance. The spectral content includes amplitude as a function of wavelength. The wavelength is swept over a predetermined range. The amplitude is determined using a photoacoustic module. The photoacoustic module includes a light emitter and an acoustic sensor. The acoustic sensor is configured to provide a sensor output corresponding to a modulated pressure detected in the tissue in response to light from the emitter. The light from the emitter corresponds to the selected wavelength. The method includes determining an amplitude difference in the spectral content as a function of the target site and the spectral content at the offset site. The method includes selecting a wavelength based on an amplitude difference.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A system comprising:
a photoacoustic module including a light emitter and an acoustic sensor, the acoustic sensor configured to provide a sensor output corresponding to a modulated pressure detected in a target tissue in response to light from the emitter;
an ablation module associated with the photoacoustic module, the ablation module having an energy discharge port configured to contact the tissue at an ablation target and configured to ablate the ablation target; and
a processor coupled to the ablation module and coupled to the photoacoustic module, the processor configured to execute an algorithm to provide an output signal indicative of ablation progress and configured to control the ablation module based on the sensor output.

2. The system of claim 1 wherein the sensor output corresponds to size, depth, or temperature of a lesion at the ablation target produced by the ablation module.

3. The system of claim 1 wherein the ablation target and the target tissue are coincident.

4. The system of claim 1 wherein the photoacoustic module provides a near real-time or real-time feedback signal corresponding to a lesion at the ablation target.

5. The system of claim 1 wherein the ablation module includes at least one of a laser, a radio frequency (RF) probe, an ultrasonic emitter, an irreversible electroporation probe, a sonoporation probe, an ablative laser, a cryoablation probe, a vapor ablation probe, a chemical ablation probe, or a DC current ablation member.

6. The system of claim 1 wherein the discharge port is configured to emit the energy directly to the target tissue.

7. A method comprising:
generating an output signal using a photoacoustic module, the output signal corresponding to a sensor output associated with a modulated pressure detected in a target tissue in response to light from an emitter of the photoacoustic module, the output signal corresponding to a physical parameter of a lesion in the target tissue;
determining an ablation energy level for an ablation module, the ablation energy level based on the output signal, the ablation energy level determined by a processor executing an algorithm, the ablation energy level corresponding to ablation energy for delivery directly to the tissue.

8. The method of claim 7 wherein the physical parameter of the lesion corresponds to at least one of size, depth, and temperature.

9. The method of claim 7 wherein determining the ablation energy level includes modulating the ablation energy level.

10. The method of claim 7 further including identifying a diseased condition of the target tissue based on the output signal.

11. The method of claim 7 further including identifying a tissue type of the target tissue based on the output signal.

12. The method of claim 7 further including determining a signal parameter corresponding to the light from the emitter, the signal parameter determined based on a tissue type for the target tissue.

13. The method of claim 12 wherein the signal parameter includes at least one of a wavelength and a frequency.

14. The method of claim 12 wherein determining the signal parameter includes conducting a biopsy, conducting a tissue extraction method, conducting a cell extraction method, conducting spectral analysis, or accessing a database.

15. The method of claim 7 wherein determining an ablation energy level includes at least one of selecting a temperature for ablation, selecting a coagulation detection threshold, selecting a timing parameter of the ablation, modulating the ablation energy level to achieve a predetermined temporal stability in the output signal, calculating a measure of viable cells, or determining a shift in a spectral content.

16. The method of claim 7 further including delivering an exogenous agent to the target tissue.

17. The method of claim 16 wherein delivering the exogenous agent includes delivering a fluorescent protein or other compound having a distinctive spectral absorbance feature.

18. A method comprising:
selecting an excitation signal parameter;
generating an output signal using a photoacoustic module, the output signal corresponding to a sensor output associated with a modulated pressure detected in a target tissue in response to light from an emitter of the photoacoustic module, the light modulated according to the excitation signal parameter; and
determining a tissue parameter corresponding to the target tissue and based on the output signal.

19. The method of claim 18 wherein the tissue parameter is associated with a tissue type.

20. The method of claim 18 wherein the tissue parameter corresponds to a dynamic tissue characteristic.
